(19) United States
(12) Patent Application Publication
(10) Pub. No.: US 2025/0366719 A1
(43) Pub. Date: Dec. 4, 2025

(54) SYSTEMS AND METHODS FOR DETERMINING SYSTEMIC VASCULAR RESISTANCE USING BIOIMPEDANCE

(71) Applicant: BaroPace, Inc., Ashland, OR (US)

(72) Inventor: Michael BURNAM, Issaquah, WA (US)

(73) Assignee: BaroPace, Inc., Ashland, OR (US)

(21) Appl. No.: 18/992,845

(22) PCT Filed: Jul. 13, 2023
(86) PCT No.: PCT/US2023/070139

§ 371 Date: Jan. 9, 2025

Related U.S. Application Data
(60) Provisional application No. 63/368,318, filed on Jul. 13, 2022.

(51) Int. Cl. A61B 5/02; A61B 5/00; A61B 5/022; A61B 5/024; A61B 5/0538

(52) U.S. Cl. CPC A61B 5/02007; A61B 5/02028; A61B 5/02225; A61B 5/024; A61B 5/0538; A61B 5/686; A61B 5/7203; A61B 5/7271; A61B 2562/0247

(57) ABSTRACT

Methods, systems, and computer-readable medium for determining a systemic vascular resistance (SVR), by receiving a systolic blood pressure (SBP) and a diastolic blood pressure (DBP) from one or more blood pressure devices, receiving a first bioimpedance from a first electrode and a second bioimpedance from a second electrode, determining a stroke volume based on a difference between the first bioimpedance and the second bioimpedance, determining a mean arterial pressure (MAP) based on the SBP and the DBP, receiving a heart rate, determining a cardiac output based on the heart rate and the stroke volume, determining a first value based on a right atrial pressure (RAP) or central venous pressure (CVP) and the map, determining a second value based on the first value and the cardiac output, and determining a SVR based on the second value and a factor.

### CROSS-REFERENCE TO RELATED APPLICATIONS

This patent application claims the benefit of U.S. Provisional Application No. 63/368,318, filed on Jul. 13, 2022, the entirety of which is incorporated by reference herein.

### TECHNICAL FIELD

Various embodiments of the present disclosure relate generally to systemic vascular resistance (SVR) determination, and, more specifically, to using bioimpedance to determine SVR.

### INTRODUCTION

The total resistance to blood flow through peripheral vascular beds has an influence on cardiac output. A rise in total peripheral resistance can raise arterial blood pressure which may reduce cardiac output. A fall in total peripheral resistance may increase cardiac output. A number of human disease states are associated with abnormal systemic vascular resistance (SVR), such as heart failure, hypertension, autoimmune disorders associated with inflammation of blood vessels, systemic infection, shock, etc.

The introduction description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for determining systemic vascular resistance (SVR) using bioimpedance.

In one aspect, an exemplary embodiment of a method for determining a systemic vascular resistance (SVR), the method comprising: receiving a systolic blood pressure (SBP) and a diastolic blood pressure (DBP) from one or more blood pressure devices; receiving a first bioimpedance from a first electrode and a second bioimpedance from a second electrode; determining a stroke volume based on a difference between the first bioimpedance and the second bioimpedance; determining a mean arterial pressure (MAP) based on the SBP and the DBP; receiving a heart rate; determining a cardiac output based on the heart rate and the stroke volume; determining a first value based on a right atrial pressure (RAP) or central venous pressure (CVP) and the MAP; determining a second value based on the first value and the cardiac output; and determining the SVR based on the second value and a factor.

In another aspect, an exemplary embodiment of a system for determining a systemic vascular resistance (SVR), the system comprising: at least one memory storing instructions; and at least one processor executing the instructions to perform a process, the at least one processor configured to: receiving a systolic blood pressure (SBP) and a diastolic blood pressure (DBP) from one or more blood pressure devices; receiving a first bioimpedance from a first electrode and a second bioimpedance from a second electrode; determining a stroke volume based on a difference between the first bioimpedance and the second bioimpedance; determining a mean arterial pressure (MAP) based on the SBP and the DBP; receiving a heart rate; determining a cardiac output based on the heart rate and the stroke volume; determining a first value based on a right atrial pressure (RAP) or central venous pressure (CVP) and the MAP; determining a second value based on the first value and the cardiac output; and determining the SVR based on the second value and a factor.

In a further aspect, an exemplary embodiment of a method for determining a systemic vascular resistance (SVR), the method comprising: determining a mean arterial pressure (MAP) based on a systolic blood pressure (SBP) and a diastolic blood pressure (DBP) received from one or more blood pressure devices; determining a stroke volume based on a difference between a first bioimpedance received from a first electrode and a second bioimpedance received from a second electrode; receiving a heart rate; determining a cardiac output based on the heart rate and the stroke volume; determining a first value based on a right atrial pressure (RAP) or central venous pressure (CVP) and the MAP; determining a second value based on the first value and the cardiac output; and determining the SVR based on the second value and a factor.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. Moreover, there are many embodiments described and illustrated herein.

FIG. 1 depicts an exemplary environment for determining a systemic vascular resistance (SVR) using bioimpedance, according to one or more embodiments.

FIG. 2 depicts a flowchart of an exemplary method for determining an SVR using bioimpedance, according to one or more embodiments.

FIG. 3 depicts an example of training a machine learning model for determining the SVR, according to one or more embodiments.

FIG. 4 depicts an example of a computing device for determining the SVR, according to one or more embodiments.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

The term “exemplary” is used in the sense of “example” rather than “ideal.” Notably, an embodiment or implementation described herein as “exemplary” is not to be construed as preferred or advantageous, for example, over other embodiments or implementations; rather, it is intended to reflect or indicate the embodiment(s) is/are “example” embodiment(s). Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. In this disclosure, the term “based on” means “based at least in part on.” The singular forms “a,” “an,” and “the” include plural referents unless the context dictates otherwise.

Embodiments of this disclosure relate generally to methods and systems for determining systemic vascular resistance (SVR) using bioimpedance. According to implementations of the disclosed subject matter, medical condition treatment and patient outcomes can be improved by monitoring SVR iteratively and/or continuously, and/or treating a medical condition based on the same. A medical condition may be treated based on monitoring and/or adjusting the administration of drugs and/or the addition or removal of body fluids, such as augmentation through infusion, or removal by drugs or dialysis, or the like, based on SVR. Patients with cardiac pacemakers form a subset of the population who suffer from a form of cardiovascular illness. For example, approximately 70% of patients with a pacemaker may have hypertension. Accordingly, the availability of real time continuous SVR measurements may be used for a diagnosis and/or therapy for such patients. The availability of SVR measurements, particularly in a continuous reliable data stream, as disclosed herein, can enhance prevention and therapy of medical conditions such as the hypertension or heart failure.

According to some embodiments, a continuous accurate method of blood pressure measurement may be implemented using a blood pressure device, as disclosed herein. Indirect measurement of cardiac output may be determined using bioimpedance for patients with pacemakers, as disclosed herein. By combining the output of both measurement techniques, pacemaker-derived bioimpedance to estimate cardiac output/stroke volume and blood pressure to estimate left or right ventricular stroke volume, a continuous measurement of SVR can be made.

As discussed herein, blood pressure (BP) may be one or both of systolic or diastolic blood pressure. Other acronyms used herein include, heart rate (HR), cardiac output (CO), and right or left ventricular stroke volume (SV). A blood pressure may indicate how much pressure a user's blood exerts against the user's artery walls when the user's heart beats (e.g., a systolic blood pressure). A blood pressure may indicate how much pressure a user's blood exerts against the user's artery walls when the user's heart is resting between beats (e.g., diastolic blood pressure). As described in detail herein, systolic blood pressure (SBP) and diastolic blood pressure (DBP) may be sensed by one or more blood pressure sensing devices (e.g., a blood pressure cuff, etc.). A first bioimpedance and a second bioimpedance may be determined from a first electrode and a second electrode, respectively, e.g., by a pacemaker. A stroke volume (SV) may be determined by the difference between the first bioimpedance and the second bioimpedance. The mean arterial pressure (MAP) may be determined based on the SBP and the DBP. A heart rate may be obtained, e.g., from the one or more blood pressure sensing devices, a pacemaker, etc. A cardiac output may be determined based on the heart rate and the SV. A first value may be determined based on a right arterial pressure (RAP) or a central venous pressure (CVP) and the MAP (e.g., by subtracting the RAP or the CVP from the MAP). A second value may be determined based on the first value and the cardiac output (e.g., by dividing the first value by the cardiac output). The SVR may be determined based on the second value and a factor (e.g., by multiplying the second value by a factor (e.g., approximately eighty)).

The term “algorithm,” as used herein, refers to a sequence of defined computer-implementable instructions, typically to solve a class of problems or to perform a computation. Terms such as “noise,” or the like, as used herein, generally encompass extraneous, irrelevant, or relatively less meaningful data, or any data that is other than a signal intended to be observed. In the case of waveforms, “noise” may include, for example, unwanted signals that are merged with the waveform signal. Terms such as “signal” or the like, a used herein, generally encompass any function that may convey information about a phenomenon. “Signals” may refer to any time varying voltage, current, or electromagnetic wave that carries information or an observable change in a quality, such as quantity, or may refer to the information itself.

As used herein, a “machine learning model” generally encompasses instructions, systems, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, layers, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or semi-supervised training may be employed. For example, supervised learning may include providing training data and labels corresponding to the training data, e.g., as ground truth. Semi-supervised approaches may include heuristic, generative, low-density, Laplacian or other like models. K-means clustering or K-Nearest Neighbors may also be used, which may be supervised or semi-supervised. Combinations of K-Nearest Neighbors and a semi-supervised cluster technique may also be used. Any suitable type of training may be used, e.g., stochastic, gradient boosted, random seeded, recursive, epoch or batch-based, etc.

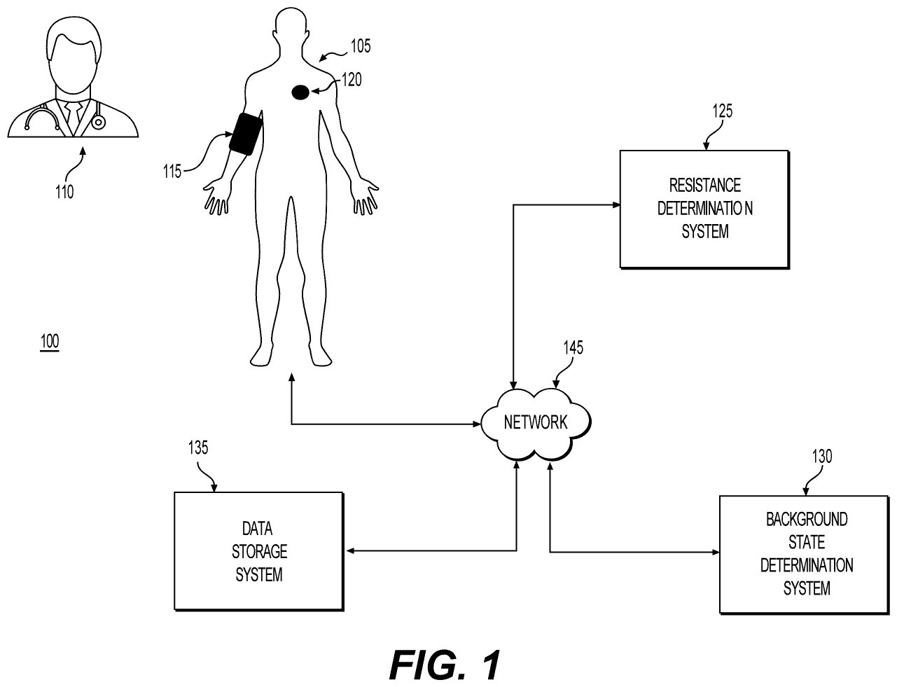

According to implementations of the disclosed subject matter, as shown in the environment 100 of FIG. 1, a user 105 and/or a medical provider 110 may operate a gold standard blood pressure device 115 and/or a pacemaker 120, the results from gold standard blood pressure device 115 and/or pacemaker 120 being transmitted via a network 145. The blood pressure devices discussed herein, e.g., gold standard blood pressure device 115, may be a blood pressure monitor that may be implemented as a standalone blood pressure monitoring device or may be incorporated in a fitness tracker, wearable device, digital watch, digital band, patch, arm cuff, a photoplethysmography (PPG) device, or the like. Pacemaker 120 may be a single chamber pacemaker with a right ventricular lead, a biventricular pacemaker, a dual chamber pacemaker, a biventricular cardio-defibrillator, or the like.

User 105 may wear gold standard blood pressure device 115 and pacemaker 120 simultaneously, or user 105 may wear one of either gold standard blood pressure device 115 or pacemaker 120 at a time. The results from gold standard blood pressure device 115 and/or pacemaker 120 may be transmitted via a network 145 between one or more of resistance determination system (hereinafter “resistance system”) 125, background state determination system (hereinafter “background state system”) 130, data storage system 135, etc. Gold standard blood pressure device 115 and/or pacemaker 120 may operate continuously, at intervals, or at the determination of user 105, medical provider 110, and/or a user. Gold standard blood pressure device 115 and/or pacemaker 120 may include one or more sensors either internal or external to the respective device. For example, pacemaker 120 may include at least one electrode. Pacemaker 120 may be a single chamber pacemaker with a right ventricular lead, a biventricular pacemaker, a dual chamber pacemaker, a biventricular cardio-defibrillator, or the like. For example, pacemaker 120 may include a biventricular pacemaker having a pacing electrode in the left ventricle (LV). In another example, pacemaker 120 may include a biventricular pacemaker having a pacing electrode in each of the LV and right ventricle (RV) for measuring a stroke volume (SV) in each ventricle for comparison to one another. In a further example, pacemaker 120 may include a dual chamber pacemaker having a pacing electrode in the RV.

According to implementations of the disclosed subject matter, blood pressure may be measured discretely such as by a single cuff inflation method of gold standard blood pressure device 115. Left or right ventricular SV may be measured using sensed bioimpedances to estimate left or right ventricular SV. At least one bioimpedance may be measured from one or more electrodes or leads of pacemaker 120. Alternatively, or in addition, bioimpedances may be measured from one or more leads of pacemaker 120 and augmented by additional skin electrodes (not depicted) connected by hardwires or wireless methods. All measurements disclosed herein may be augmented, trended, and/or individualized using artificial intelligence, such as machine learning. According to an implementation, skin sensing techniques may be augmented beyond the capabilities of standard electrocardiogram (EKG) electrodes using solid-state sensing elements, either silicon-based, metal film, non-aqueous polymeric plastic with immobilized ions, or field effect transistor methods.

According to embodiments disclosed herein, resistance system 125 may be configured to determine an SVR. Resistance system 125 may be configured to obtain at least one of a SBP, a DBP, two or more bioimpedances (e.g., a first bioimpedance, a second bioimpedance, etc.), a heart rate, etc. from aspects of environment 100. Based on the data obtained, resistance system 125 may be configured to determine at least one of a SV, a MAP, a CO, and more. Resistance system 125 may be configured to transmit data (e.g., the obtained data, the determined data, etc.) to any suitable aspect of environment 100. According to implementations of the disclosed subject matter, SVR may be determined by resistance system 125 using blood pressure and one or more leads of pacemaker 120 to estimate right or left ventricular SV using bioimpedance. Artificial intelligence such as machine learning may be used to output the SVR or to output one or more modified or corrected blood pressures, SV estimates, bioimpedances, or the like.

The machine learning model may analyze data received from user 105, provider 110, gold standard blood pressure device 115, pacemaker 120, data storage system 135, and/or any other person, entity, or device. In one example, a digital scale may be used as an input to a machine learning module (e.g., to correct the bioimpedance data for fluid loss). It should be appreciated that a change in body weight in a patient experiencing heart failure may correspond to an increase in body fluid (e.g., due to a heart condition). If a total body bioimpedance rises by a predetermined percentage, and a body weight of the patient rises by a predetermined amount of (e.g., in kilograms), an actual measurement (e.g., in milliliters) of fluid accumulated during any one time period may be estimated, such as by using the known density of water (e.g., one gram per milliliter). In another example, data from gold standard blood pressure device 115 (e.g., blood pressure data) and/or data from pacemaker 120 (pacemaker data) may be input into the machine learning model. The trained machine learning model may correlate a SBP, a DBP, two or more bioimpedances (e.g., a first bioimpedance, a second bioimpedance, etc.), a heart rate, a SV, a MAP, a CO, etc. to determine SVR. The machine learning model may output a SVR.

In various embodiments, a processor or storage component (e.g., data storage system 135), gold standard blood pressure device 115, and/or pacemaker 120 may generate, store, train, or use the machine learning model and/or may include instructions associated with the machine learning model, e.g., instructions for generating the machine learning model, training the machine learning model, using the machine learning model, etc. For example, blood pressure measured using gold standard blood pressure device 115 may be transmitted, via a Bluetooth protocol, to processor associated with pacemaker 120. The processor may receive the measured blood pressure to output the SVR or to output one or more modified or corrected blood pressures, SV estimates, bioimpedances, or the like. The processor or one or more other processors may apply the modified or corrected values to more accurately determine the SVR.

In some embodiments, a system or device other than gold standard blood pressure device 115 or pacemaker 120 may be used to generate and/or train the machine learning model. For example, such a system may include instructions for generating the machine learning model, the training data and ground truth, and/or instructions for training the machine learning model. Training data may include one or more of historical blood pressures, historical bioimpedances, historical SBPs, historical DBPs, historical MAPs, historical RAPs, historical CVPs, historical first values, historical second values, historical SVRs, and more from a plurality of users. A resulting trained machine learning model may then be provided to pacemaker 120 or a component associated with pacemaker 120 such that the trained machine learning model can output an SVR and/or one or more modified or corrected blood pressures, SV estimates, bioimpedances, etc.

Generally, a machine learning model includes a set of variables, e.g., layers, nodes, neurons, filters, weights, biases, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, adaptive moment estimation (“ADAM”), etc. Training may be conducted with or without sample and/or class weighting. In some embodiments, a portion of the training data may be withheld during training and/or used to validate the trained machine-learning model, e.g., compare the output of the trained model with the ground truth for that portion of the training data. The training of the machine learning model may be configured to cause the machine learning model to learn associations between (i) gold standard data and/or PPG device data and (ii) gravitational effects based on device positioning, such that the trained machine learning model is configured to determine an output (e.g., corrected PPG device data) in response to the input data based on the learned associations. For example, the machine learning model may receive PPG device data points (e.g., blood pressure) associated with a particular arm positioning, which the machine learning model may be trained to correct based on the calibration factor applied to the arm positioning.

In various embodiments, the variables of a machine learning model may be interrelated in any suitable arrangement in order to generate the output. For example, in some embodiments, the machine learning model may include architecture that is configured to identify, isolate, and/or extract features, geometry, and or structure in input data. For example, the machine learning model may include one or more convolutional neural networks (“CNN”) configured to identify features in the signal-processed data, and may include further architecture, e.g., a connected layer, neural network, etc., configured to determine a relationship between the identified features in order to determine a location in the signal-processed data.

According to embodiments disclosed herein, background state system 130 may be configured to filter (e.g., reduced, modified, and/or removed) one or more forms of noise (e.g., background states) found in one or more signals, e.g., bioimpedance, blood pressure, etc. The one or more signals may be generated using gold standard blood pressure device 115 and/or pacemaker 120. A noise reduction algorithm may be used by background state system 130 to filter noise. The type of noise reduction algorithm may depend on the type of noise in the data, the type of data, or the like. The noise reduction algorithm may be automatically selected and/or applied or may be selected and/or applied based on user input. A type of noise may include, but is not limited to, high frequency noise, movement noise, and/or any other form of noise. A plurality of noise reduction algorithms may be used to filter noise for a given signal. According to embodiments disclosed herein an amplification may be applied to amplify a signal generated at gold standard blood pressure device 115 and/or pacemaker 120. Such signal amplification may be performed prior to, in conjunction with, or post filtering the signal for noise.

Techniques disclosed herein may be implemented using communication (e.g., wireless communication), such as one-way communication, with a remote server for remote data storage, data evaluation and processing, communication with a third party (e.g., a health care provider). The remote server may be a cloud component. A base unit with communication (e.g., wireless communication) capability may be in communication with a pacemaker (e.g., pacemaker 120) and a blood pressure sensing device (e.g., gold standard blood pressure device 115), and may include processing software. The base unit may be integrated in one or more blood pressure devices (e.g., a smartwatch) or pacemaker.

In some embodiments, the network 145 may connect one or more components of the environment 100 via a wired connection, e.g., a USB connection between gold standard blood pressure device 115 and pacemaker 120. In some embodiments, the network 145 may connect one or more aspects of the environment 100 via an electronic network connection, for example a Bluetooth connection, a wide area network (“WAN”), a local area network (“LAN”), personal area network (“PAN”), or the like. In some embodiments, the electronic network connection includes the Internet, and information and data provided between various systems occurs online. “Online” may mean connecting to or accessing source data or information from a location remote from other devices or networks coupled to the Internet.

Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

According to embodiments, environment 100 may be a closed loop such that no external network connection may be necessary to implement the techniques disclosed herein. The closed loop maybe used to provide a real-time automatic method that is self-contained and not dependent upon linkage to a remote server containing additional software, often referred to as “edge computing.” The method is also suitable for transmission to the cloud to allow for an interface with conventional electronic health records and other data analysis and reporting processes.

In such a closed loop system, as discussed herein, a blood pressure and/or a bioimpedance (e.g., a first bioimpedance, a second bioimpedance, etc.) may be transmitted over a Bluetooth connection. Pacemaker 120 may be associated with a processor that may apply a received and/or determined blood pressure, bioimpedance, heart rate, cardiac output, stroke volume, etc. to determine a systemic vascular resistance (e.g., using a machine learning model). Accordingly, the connections within the environment 100 can be wireless, wired, or be any other suitable connection, or any combination thereof.

In some embodiments, the data storage system 135 may store the data from and/or provide data to various aspects of the environment 100. Data storage system 135 may include a server system, an electronic medical data system, computer-readable memory such as a hard drive, flash drive, disk, etc. In some embodiments, data storage system 135 includes and/or interacts with an application programming interface for exchanging data to other systems, e.g., one or more of the other components of the environment 100. Data storage system 135 may include and/or act as a repository or source for data from gold standard blood pressure device 115, data from pacemaker 120, medical history and/or diagnoses for user 105, machine learning data, and/or other forms of data. Data storage system 135 may be external to or may be a part of gold standard blood pressure device 115 or pacemaker 120.

Traditional continuous SVR measurements may be performed using an invasive approach, such as an arterial cannula. Traditional noninvasive measurements are serially generated several minutes apart (not continuously). During such noninvasive measurements, the compressive effects of a traditional blood pressure cuff may become progressively painful and influence the results by inducing peripheral artery spasm. Traditional system cannot calculate continuous SVR without a calibrated means to determine continuous blood pressure. Traditional external bioimpedance measurement devices suffer from multiple sources of inaccuracy, including, but not limited to, variables such as a given physical model (e.g., electrode array), cross sectional area variation (e.g., person to person variation in total electrical volume), ethnicity, body hydration, age, level of body fat, among other variables. Equations for each population or user can be required with traditional bioimpedance devices as generic equations (e.g., manufacturer provided equations) can result in overestimation or under estimation. Although expensive peripheral bioimpedance devices have attempted to be used for the estimation of a range of hemodynamics including cardiac output, SVR, and total body fluid content, the large measurement variability encountered has limited their use to estimation of total body fat, which estimations are also inaccurate due to the reasons described above.

Traditional bioimpedance measurement systems measure bioimpedance to estimate thoracic fluid content without accounting for accompanying hemodynamic variables such as stroke volume. Such systems suffer from at least two drawbacks: the total area sampled between a pacemaker lead tip and the pacemaker can span a small percentage of the thoracic cavity without sampling the areas of the lungs most likely to accumulate fluid in heart failure. In addition, such systems cannot distinguish between heart failure fluid and fluid accumulation for other reasons such as, for example, pneumonia and bronchitis. Implementations disclosed herein use estimated stroke volumes based on bioimpedance in conjunction with a blood pressure measurement and heart rate (e.g., from a pacemaker or other heart rate detection device) to calculate SVR. The combination of the bioimpedance based stroke volume and blood pressure are used to implement the continuous SVR measurement techniques disclosed herein.

As discussed below, SVR may be determined based on blood pressure and a cardiac output determined using bioimpedance. One or more blood pressure sensing devices may output an SBP and a DBP. Further, bioimpedance based measurements using pacemaker leads may be used to estimate a stroke volume. MAP may be determined based on Equation 1 (see below), using the SBP and DBP. A heart rate may be determined using a pacemaker or other heart rate sensing device. A cardiac output may be determined by multiplying the heart rate by the stroke volume estimated using the differences in bioimpedance, as discussed herein. A CVP, as further discussed below, or RAP may be subtracted from the MAP. The resulting value may be divided by the cardiac output. The resulting value may be multiplied by a factor (e.g., approximately 80) to generate an estimated SVR.

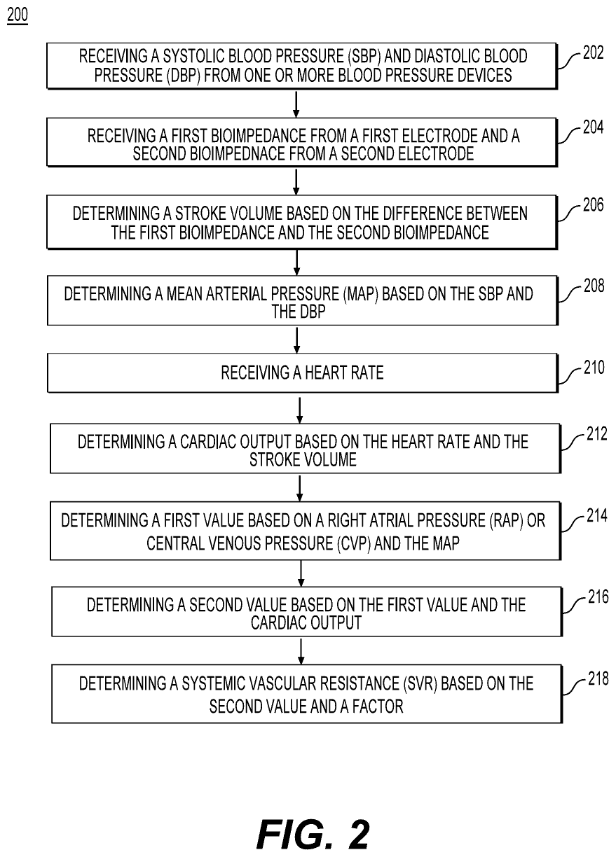

FIG. 2 shows a flow chart 200 for determining an SVR using bioimpedance. At step 202, an SBP and a DBP may be received from one or more blood pressure measuring devices. According to implementations of the disclosed subject matter, continuous blood pressures or serial discrete blood pressure measurements may be obtained using a blood pressure device, as disclosed herein. SV may be determined based on difference in bioimpedance across two heart chambers during a heartbeat cycle. The blood pressures may be used to determine real-time continuous SVR or serial discrete SVR measurements in patients by applying SV using bioimpedance, for diagnosing and treating conditions associated with abnormal SVR. The differences between discrete and continuous measurements include, but are not limited to sampling continuously or high frequency discrete sampling (e.g., higher resolution in time) carries higher information content, open loop verses closed control has different entry/exits (e.g., in software), multiple masters and transaction initiators in Internet of Things (IOT), and/or aggregation and actuation can happen in a closed loop and/or a local plus remote system.

Continuous or a series of discrete measurements can be used for a closed loop system of control and treatment. As the volume of measurements over time increases, differences in response between individuals may lead to personalized models of treatment for each patient. In addition, the differences in response of the same patient over time can be analyzed to identify changes in a patient's condition and/or treatment plans, accordingly. These changes can be triggered automatically by the application of functions designed specifically to identify changes over a programmable set of threshold parameter values. Additionally, once a closed loop control system is established, automatic changes may be effected by the system (e.g., intervention by a healthcare professional to make changes may become optional).

Additionally, intervention by a healthcare professional to alter one or more parameters of the system or a treatment plan may be effected remotely using a secure communication channel without the need for a physical visit to the provider's facility. Additionally, intervention or evaluation by a healthcare professional may be initiated by the system autonomously as opposed to being initiated by the patient or as a routine/periodic check by the healthcare professional.

Still referring to FIG. 2 at step 204, a first bioimpedance measurement from a first electrode (e.g., a first pacemaker electrode) and a second bioimpedance measurement from a second electrode (e.g., a second pacemaker electrode) may be received. In some embodiments, SVR may be estimated using SVs determined using bioimpedance differences. The bioimpedance differences may be determined using, for example, a single chamber pacemaker with a right ventricular lead, a dual chamber pacemaker, a biventricular pacemaker, a biventricular cardio-defibrillator, or the like with communication (e.g., wireless communication) capability. One or more blood pressure sensing devices may be used to determine SBP and DBP. For example, the one or more blood pressure sensing devices may be cleared (Food and Drug Administration (FDA)-cleared, approved for use in the European Union, or approved in other locales) patient operated blood pressure cuffs with communication capability (e.g. wireless communication capability).

Two different electrodes (e.g., pacemaker leads) may be connected to two heart chambers, respectively. The peak bioimpedance of a first chamber may be sensed by the first electrode, as the chamber fills with blood (e.g., a peak value from a distribution of bioimpedances during a heartbeat cycle in the first chamber). The peak bioimpedance may be converted to a diastolic volume. Bioimpedances described herein may be measured from a lead tip to a pacemaker component (e.g., a metallic can component). Such calculations may be performed one chamber at a time. For example, the pacemaker component may be a reference (e.g., ground) and may be applied relative to a current from an electrode tip. According to an example implementation, if a pacemaker lead includes more than one pacing electrode in an array, a single electrode may be used at a given time. For example, an electrode farthest from the pacemaker component may be used to determine bioimpedance. Subsequently, the blood from the first chamber may be output to the second chamber (e.g., during a same heart beat cycle). The peak bioimpedance of the second chamber may be sensed by the second electrode, as the second chamber fills with blood (e.g., a peak value from a distribution of bioimpedances during the heart beat cycle in the second chamber). The peak bioimpedance of the second chamber may be converted to a systolic volume.

At step 206, the difference between the first bioimpedance and second bioimpedance may be used to determine an SV, as disclosed herein. A first heart chamber may fill with blood and all or a proportion of the blood may empty to a second chamber. The difference in volume between the filled first chamber and the filled second chamber may be used for the calculation of a cardiac stroke volume which may be used to calculate cardiac output by multiplying stroke volume by the heart rate (Cardiac Output=Heart Rate×Stroke volume). For example, right atrial (RA) end diastolic volume may be estimated based on bioimpedance sensed by a first pacemaker lead. A right ventricle (RV) end systolic volume may be estimated based on bioimpedance sensed by a second pacemaker lead. The diastolic volume minus the systolic volume may be the RV stroke volume (SV) or may be proportional to RV SV, which may then be corrected by a constant. As another example, RA end diastolic volume may be estimated based on bioimpedance sensed by a first pacemaker lead. Left ventricle (LV) end systolic volume may be calculated based on bioimpedance sensed by a second pacemaker lead. The diastolic volume minus the systolic volume may be the LV SV, or may be proportional to LV SV and is corrected by a constant.

According to implementations of the disclosed subject matter, SVR may be estimated using SVs that are determined using bioimpedance differences. SV (e.g., actual volume of blood per heat beat) may include a difference between left and/or right ventricular filled volume before ejection and a remaining volume after ejection, which may be expressed as LV (or RV) end diastolic volume (e.g., filled volume or LVedv), and LV (or RV) end systolic volume (e.g., LVesv). For example, the L Vesv may include a remaining volume after the blood is ejected through the aortic or pulmonic valve.

In some embodiments, bioimpedances may be used to estimate (e.g., correlate with actual) LVedv and LVesv, e.g., by measuring the bioimpedance across a fluid volume of interest as the difference between two electrical poles. For example, for the RV, a pacing electrode at the tip of the RV may be one pole, and an additional vector “away” from the RV tip may be utilized for comparison. In one example, the pacing electrode at the tip of the RV may be positioned relatively far (e.g., a predefined minimum distance) from the RV tip, such as proximate to a base of a patient's neck, either internally or on the skin. The bioimpedance across the two poles may vary with each heartbeat, such as proportionally to the true SV. Baseline readings with the patient at rest may be obtained using one or more of the devices described herein, and changes in SVR during a period (e.g., a day) may be followed and/or monitored with particular events (e.g., exercise) noted by the patient and/or sensed by the device (e.g., via an accelerometer). The correlations of triggers causing changes in SV may be determined. SV multiplied by heart rate (HR) may define a cardiac output (CO), which may be reported as a trend change.

In other examples, a calibrated version may be determined, such as via a gold standard (or “actual”) measurement of SV for comparison. An actual SV measurement may be taken invasively, such as at a time of cardiac catheterization, for a specific case of LV assist devices which measure SV, and/or any approved non-invasive device that measures one or more of SV or CO. Comparison to an actual SV determination may allow a backward calibration of the relative bioimpedance measurement in mamps (LVedv-LVesv) to the actual gold standard measurement in milliliters of blood ejected per beat.

Still referring to FIG. 2 at step 208, a MAP may be determined based on the SBP and the DBP (e.g., as shown in Equation 1). For example, MAP may be determined by the following Equation 1 using systolic blood pressure (SBP) and diastolic blood pressure (DBP): MAP=SBP+2 (DBP). At step 210, a heart rate may be received from a heart rate measuring device (e.g., pacemaker 120). A heart rate may be determined using a pacemaker or other heart rate sensing device. At step 212, a cardiac output may be determined based on the heart rate and the SV (e.g., by multiplying the heart rate by the SV). According to an implementation, cardiac output may be determined for patients with cardiac pacemakers using bioimpedance. According to implementations of the disclosed subject matter, cardiac output may be determined based on differences in bioimpedance calculated at two heart chambers using one or more formulas. The difference between the calculated diastolic volume and the systolic volume, calculated using the respective bioimpedances, multiplied by the heart rate, may be the cardiac output of the heart. The change in bioimpedance may occur over time and can be phasic with a heartbeat as blood volume changes. Accordingly, the bioimpedance values discussed herein may expressed as derivatives. As another example, a method for calculating cardiac output based on bioimpedance is disclosed in U.S. Pat. No. 10,918,858 B2, issued Feb. 16, 2021, which is incorporated herein by reference in its entirety.

At step 214, a first value may be determined based on one of (i) the RAP or (ii) the CVP, and the MAP (e.g., by subtracting the RAP or the CVP from the MAP). RAP may be directly measured (e.g., invasively) and may be in the range of approximately 8 mmHg to 12 mmHg as a mean number. RAP may be estimated clinically. During a clinical estimation, a patient may be placed supine on their side (e.g., left side) with the head at approximately 45 degrees. In this positon, the venous pulsations in the neck may be noted in cm from the level of the clavicle. Alternatively, the RAP may be estimated to be approximately 10 mmHg. Alternatively, or in addition, RAP may be measured indirectly using echocardiography. CVP may be determined either directly by inserting a catheter into the body of the right atrium or may be indirectly estimated. The magnitude of CVP may be small compared to one or more other measurement parameters. Accordingly, an indirectly measured CVP may be estimated by assigning a mean value of 10 mmHg. The estimated CVP may be adjusted based on visual estimation of a Jugular Venous Pressure (e.g., if such Jugular Venous Pressure suggests a higher CVP value).

Still referring to FIG. 2 at step 216, a second value may be determined based on the first value and the cardiac output, e.g., by dividing the first value (determined at step 214) by the cardiac output (determined at step 212). At step 218, an SVR may be determined based on the second value and a factor (e.g., by multiplying the second value by approximately 80). The two derived measurements—blood pressure (systolic and diastolic) and cardiac output/stroke volume—may be combined to determine an estimated SVR by subtracting the right atrial pressure (RAP) or central venous pressure (CVP) from the mean arterial pressure (MAP) (see step 214), divided by the cardiac output (see step 216), and multiplied by a factor (e.g., approximately eighty). Normal SVR may be approximately 700 dynes/seconds/cm-5 to approximately 1,500 dynes/seconds/cm−5.

The sensitivity and specificity of the SVR determination technique disclosed herein may be further augmented by using a sensor device to detect background states (e.g., noise, motion, interference signals, etc.), e.g., by background state system 130. For example, a sensor device (e.g., a peripheral sensor device) such as a wrist bracelet, ankle bracelet, smartwatch, or the like may be used to determine background states. Such sensor device based measurements may expand the total volume of available measurements. Traditional devices for measurement of bioimpedance (e.g., such as the Optivol® system marketed by Medtronic, Inc.) suffer from a limited special vector volume that is confined to the area between the pacemaker lead tips and the pacemaker metal can. By adding additional spatial volume to the measurements discussed herein, such as by including a sensing device (e.g., a wireless bioimpedance-sensing bracelet) to the sensor network, artifact may be reduced and measurement sensitivity and specificity may proportionately increase. Such traditional systems (e.g., the Optivol® system) also suffer from false positive total thoracic compartment fluid estimations (e.g., for diagnosis of heart failure) when the source of the fluid is confined to the lungs due to an inflammatory state and/or infection rather than heart failure. Such false positives are generally a drawback of traditional pacemaker lead-derived bioimpedance measurements, especially systems where the spatial vectors are limited to the area over or around the left lung tissue directly adjacent (e.g., as in the Optivol® system).

The sensor device may have wireless communication capabilities to communicate with a processor (e.g., a wearable device processor or related processor) to provide both a more stable background state not limited to the bioimpedance volume confined between pacemaker lead tips and a pacemaker can. Such a background state(s) determined using a sensor device may be used in addition to or instead of, for example, using a single sensing element on a pacemaker. According to this implementation, the background state may be used to isolate the contribution of thoracic fluid content as a separate variable, and to remove it as an artifact from a SVR determination. Stated differently, thoracic fluid may include a form of noise or background state which may change, such as in response to the patient experiencing heart failure and the lungs filling with fluid. Thoracic fluid impedance may be estimated by measuring a bioimpedance across the thorax with the diaphragm up (e.g., lungs empty) and diaphragm down (e.g., lungs filled). The bioimpedance with electrodes surrounding a thorax with the bottom level at a level of the diaphragm may be measured with the lungs filled and empty. Total thoracic impedance may be estimated by the total bioimpedance with the lungs filled. Contributions to the total bioimpedance by a lung compartment may be determined, and particularly removed, by subtracting the bioimpedance with the diaphragm down) from the bioimpedance with diaphragm up.

Accordingly, sensed data from a sensor device may be used to remove background state artifacts from one or more measurements disclosed herein, when determining an SVR. A reduction in the background noise or any artifact (e.g., motion artifact) may be implemented using systems and/or components intrinsic to a blood pressure device without using a sensor device. However, it will be understood that adding a sensor device (e.g., an additional vector or area of measurement) to a given ground may provide enhanced artifact correction.

According to implementations of the disclosed subject matter, a time derivative of the bioimpedance change may be used as an indirect method of estimated stroke volume. The measurement of SVR in the manner disclosed herein without a pacemaker may be performed by extending the volume of impedance measured from the surface of the body multiple skin electrodes as previously described, or by augmenting surface electrode approach using bracelets and/or anklets including electrodes interconnected via a mode of communication (e.g., wireless communication via, for example, Bluetooth).

According to implementations of the disclosed subject matter, data analysis, trends, calibration factors, or one or more other items disclosed herein may be output by and/or modified by a machine learning model, a rules-based non AI method, a neural method, or other means of artificial intelligence processing. The machine learning model may be trained to modify or correct such items based on individual user data, cohort user data, historical data, changes in user states, trends, or the like. The machine learning model may be implemented as a subroutine and may process data derived from SVR measurements for additional measurement correction. The machine learning model may be implemented using the base unit disclosed herein. According to implementations of the disclosed subject matter, wireless transmission techniques for transmitting and/or receiving items disclosed herein may be implemented. Sensed data, estimated data, blood pressure values, SV, SVR, etc. may be wirelessly transmitted and/or received via a network, as further described herein. Such data may be stored locally or at a remote storage or memory such as in a cloud component. The cloud component or other storage or memory may be implemented using security protocols configured to mitigate loss of data, to mask data, to encrypt data, or the like.

The techniques disclosed herein may be implemented via a bench top integration and testing of the components disclosed herein. A prototype using a continuous measurement format or frequency discrete measurements may be tested in an anesthetized animal model, and then validated in humans in a cardiac catheterization laboratory in parallel with regular diagnostic catheterization carried out in consenting patient subjects. According to implementations, SVR may be determined using blood pressures sensed using one or more blood pressure devices and one or more pacemaker leads to estimate right or left ventricular SV using bioimpedance where the sensitivity of the bioimpedance measurement may be augmented by one or more additional measurement sites using skin electrodes (e.g., hard-wired skin electrodes and/or wireless skin electrodes). The skin electrodes may be solid-state sensing elements, and may be one or more of silicon-based, metal film, non-aqueous polymeric plastic with immobilized ions, field effect transistor methods, and/or the like.

According to implementations of the disclosed subject matter, blood pressures may be provided non-invasively using one or more continuous or discrete measurement devices including but not limited to, cuff-based, cyclic, sphygmomanometer type blood pressure measuring, or other applicable devices. Component signals such as bioimpedance measurements or other measurements disclosed herein may be pre-processed and/or post processed to include motion artifact reduction, band pass filtering (e.g., for noise reduction, stabilization, amplification, etc.), position correction, 5G (fifth generation) correction, and/or the like. Such component signals may be analyzed as discrete time/event data, and/or continuous domain data. One or more data disclosed herein (e.g., calculations, analysis, measurements, signals, estimates, etc.) may be maintained off site, such as at a cloud component with secure wireless communication and/or storage.

Techniques disclosed herein may be implemented in a closed-loop therapeutic system to diagnose and/or treat medical conditions including, but not limited to, hypertension and heart failure. The closed loop system may be implemented in real-time and/or autonomously utilizing all or part of the data discussed herein, including but not limited to SVR, and may include real-time patient interaction with one or more control algorithm(s). Data that is output, calculated, sensed, determined, or otherwise discussed herein, such as SVR, or any other variables (e.g., hemodynamic variables) including but not limited to HR, CO, SV, pulse oximetry, cardiac rhythm, bioimpedance, non-aqueous in vitro diagnostic blood level testing methods such as for glucose, etc. may be provided to and/or used by any applicable diagnostic or therapeutic system, such as a remote or mobile patient treatment device, an entity, a system, a component, or the like or a combination thereof.

Techniques disclosed herein may be used to regulate a cardiac pacemaker, cardio-defibrillator, combination device, or other bodily implant for the diagnosis or treatment of disease. Said regulation may be implemented in a real-time automatic closed loop system. Alternatively, or in addition, such regulation may be implemented using a component in a decision chain that is not a closed loop in design, with manual data entry or control points operated by an operator such as a user, a health care provider, or the like (e.g., on site or remotely).

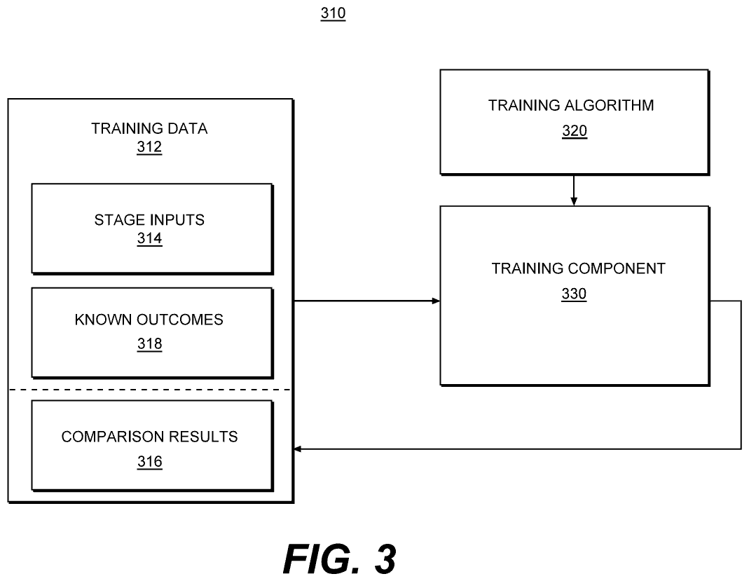

One or more implementations disclosed herein may be applied by using a machine learning model, another AI system such as a neural network, or a non-AI rules-based system. For example, a machine learning model may be used to determine a state machine and/or a next state. As shown in flow diagram 310 of FIG. 3, training data 312 may include one or more of stage inputs 314 and known outcomes 318 related to a machine learning model to be trained. The stage inputs 314 may be from any applicable source including an input or system discussed herein (e.g., an output from a step of FIG. 2). The known outcomes 318 may be included for machine learning models generated based on supervised or semi-supervised training. An unsupervised machine learning model might not be trained using known outcomes 318. Known outcomes 318 may include known or desired outputs for future inputs similar to or in the same category as stage inputs 314 that do not have corresponding known outputs.

The training data 312 and a training algorithm 320 may be provided to a training component 330 that may apply the training data 312 to the training algorithm 320 to generate a machine learning model. According to an implementation, the training component 330 may be provided comparison results 316 that compare a previous output of the corresponding machine learning model to apply the previous result to re-train the machine learning model. The comparison results 316 may be used by the training component 330 to update the corresponding machine learning model. The training algorithm 320 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as the flows and/or process discussed herein (e.g., in FIGS. 1-3), etc., may be performed by one or more processors of a computer system, such any systems or devices used to implement the techniques disclosed herein. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

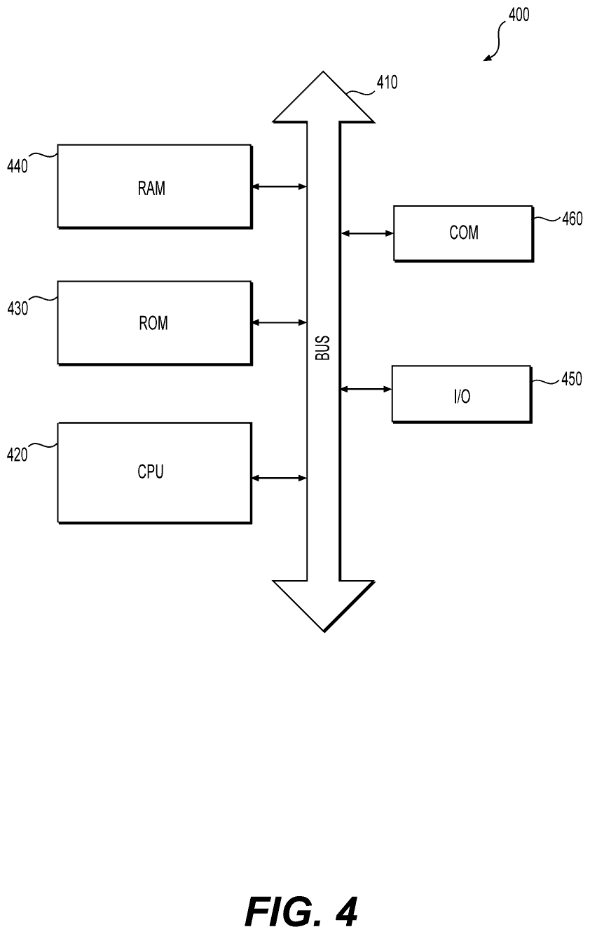

FIG. 4 depicts an example system 400 that may execute techniques presented herein. FIG. 4 is a simplified functional block diagram of a computer that may be configured to execute techniques described herein, according to exemplary embodiments of the present disclosure. Specifically, the computer (or “platform” as it may not be a single physical computer infrastructure) may include a data communication interface 460 for packet data communication. The platform may also include a central processing unit (“CPU”) 420, in the form of one or more processors, for executing program instructions. The platform may include an internal communication bus 410, and the platform may also include a program storage and/or a data storage for various data files to be processed and/or communicated by the platform such as ROM 430 and RAM 440, although the system 400 may receive programming and data via network communications. The system 400 also may include input and output ports 450 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

As discussed herein, a memory may include a device or system that is used to store information for immediate use in a computer or related computer hardware and digital electronic devices. Contents of memory can be transferred to storage (e.g., via virtual memory). Memory may be implemented as semiconductor memory, where data is stored within memory cells built from MOS transistors on an integrated circuit. Semiconductor memory may include volatile and/or non-volatile memory. Examples of non-volatile memory include flash memory and read-only memory (ROM), programmable read-only memory (PROM), erasable programmable read-only memory (EPROM), electrically erasable programmable read-only memory (EEPROM) memory, and the like. Examples of volatile memory include primary memory such as dynamic random-access memory (DRAM) and fast CPU cache memory such as static random-access memory (SRAM).

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A method for determining a systemic vascular resistance (SVR), the method comprising:
receiving a systolic blood pressure (SBP) and a diastolic blood pressure (DBP) from one or more blood pressure devices;
receiving a first bioimpedance from a first electrode and a second bioimpedance from a second electrode;
determining a stroke volume based on a difference between the first bioimpedance and the second bioimpedance;
determining a mean arterial pressure (MAP) based on the SBP and the DBP;
receiving a heart rate;
determining a cardiac output based on the heart rate and the stroke volume;
determining a first value based on a right atrial pressure (RAP) or central venous pressure (CVP) and the MAP;
determining a second value based on the first value and the cardiac output; and
determining the SVR based on the second value and a factor.

2. The method of claim 1, wherein the one or more blood pressure devices are selected from one or more of a cuff-based device, a cyclic device, or a sphygmomanometer.

3. The method of claim 1, wherein the one or more blood pressure devices are configured to detect one or both of the SBP or the DBP continuously.

4. The method of claim 1, wherein at least one of the first electrode or the second electrode is a pacemaker electrode.

5. The method of claim 1, wherein the first bioimpedance is a first heart chamber bioimpedance and the second bioimpedance is a second heart chamber bioimpedance.

6. The method of claim 1, wherein the first bioimpedance and the second bioimpedance correspond to a same heartbeat cycle.

7. The method of claim 1, further comprising a machine learning model configured to generate a machine learning output to individualize at least one of the first bioimpedance, the second bioimpedance, the SBP, the DBP, the MAP, the RAP, the CVP, the first value, the second value, or the SVR.

8. The method of claim 7, wherein the machine learning model is trained using training data including one or more of historical blood pressures, historical bioimpedances, historical SBPs, historical DBPs, historical MAPs, historical RAPs, historical CVPs, historical first values, historical second values, or historical SVRs.

9. The method claim 1, wherein one or more of the first bioimpedance, the second bioimpedance, the SBP, the DBP, the MAP, the RAP, the CVP, the first value, the second value, or the SVR are filtered for one or more of noise reduction, stabilization, or amplification.

10. A system for determining a systemic vascular resistance (SVR), the system comprising:
at least one memory storing instructions; and
at least one processor executing the instructions to perform a process, the at least one processor configured to:
receiving a systolic blood pressure (SBP) and a diastolic blood pressure (DBP) from one or more blood pressure devices;
receiving a first bioimpedance from a first electrode and a second bioimpedance from a second electrode;
determining a stroke volume based on a difference between the first bioimpedance and the second bioimpedance;
determining a mean arterial pressure (MAP) based on the SBP and the DBP;
receiving a heart rate;
determining a cardiac output based on the heart rate and the stroke volume;
determining a first value based on a right atrial pressure (RAP) or central venous pressure (CVP) and the MAP:
determining a second value based on the first value and the cardiac output; and
determining the SVR based on the second value and a factor.

11. The system of claim 10, wherein the one or more blood pressure devices are selected from one or more of a cuff-based device, a cyclic device, or a sphygmomanometer.

12. The system of claim 10, wherein the one or more blood pressure devices are configured to detect one or both of the SBP or the DBP continuously.

13. The system of claim 10, wherein at least one of the first electrode or the second electrode is a pacemaker electrode.

14. The system of claim 10, wherein the first bioimpedance is a first heart chamber bioimpedance and the second bioimpedance is a second heart chamber bioimpedance.

15. The system of claim 10, wherein the first bioimpedance and the second bioimpedance correspond to a same heartbeat cycle.

16. The system of claim 10, further comprising a machine learning model configured to generate a machine learning output to individualize at least one of the first bioimpedance, the second bioimpedance, the SBP, the DBP, the MAP, the RAP, the CVP, the first value, the second value, or the SVR.

17. The system of any one of claims 16, wherein the machine learning model is trained using training data including one or more of historical blood pressures, historical bioimpedances, historical SBPs, historical DBPs, historical MAPs, historical RAPs, historical CVPs, historical first values, historical second values, or historical SVRs.

18. The system of claim 10, wherein one or more of the first bioimpedance, the second bioimpedance, the SBP, the DBP, the MAP, the RAP, the CVP, the first value, the second value, or the SVR are filtered for one or more of noise reduction, stabilization, or amplification.

19. A method for determining a systemic vascular resistance (SVR), the method comprising:
determining a mean arterial pressure (MAP) based on a systolic blood pressure (SBP) and a diastolic blood pressure (DBP) received from one or more blood pressure devices;
determining a stroke volume based on a difference between a first bioimpedance received from a first electrode and a second bioimpedance received from a second electrode;
receiving a heart rate;
determining a cardiac output based on the heart rate and the stroke volume;
determining a first value based on a right atrial pressure (RAP) or central venous pressure (CVP) and the MAP;
determining a second value based on the first value and the cardiac output; and
determining the SVR based on the second value and a factor.

20. The method of claim 19, further comprising:
generating a machine learning output by a machine learning model to individualize at least one of the first bioimpedance, the second bioimpedance, the SBP, the DBP, the MAP, the RAP, the CVP, the first value, the second value, or the SVR.
